(19) United States
(12) Patent Application Publication
(10) Pub. No.: US 2021/0346662 A1
(43) Pub. Date: Nov. 11, 2021

(54) TAMPER EVIDENT TRANSDERMAL PATCH

(71) Applicant: KINDEVA DRUG DELIVERY L.P., St. Paul, MN (US)

(72) Inventors: Nicholas T. Gabriel, St. Paul, MN (US); Timothy A. Peterson, St. Paul, MN (US); Ronald D. Jesme, St. Paul, MN (US); James P. Dizio, St. Paul, MN (US); Scott A. Burton, St. Paul, MN (US); Kevin L. Puckett, St. Paul, MN (US); Kraig D. Kroells, St. Paul, MN (US)

(21) Appl. No.: 17/286,846

(22) PCT Filed: Oct. 14, 2019
(86) PCT No.: PCT/IB2019/058733

§ 371 Date: Apr. 20, 2021

Related U.S. Application Data
(60) Provisional application No. 62/749,377, filed on Oct. 23, 2018.

(51) Int. Cl. A61M 35/00; A61M 37/00; A61K 9/70

(52) U.S. Cl. CPC A61M 35/10; A61M 37/0015; A61K 9/7092; A61M 2230/65; A61M 2205/3561; A61M 2205/6054; A61M 2037/0023

(57) ABSTRACT

Transdermal patch having at least one pharmaceutically active ingredient and a tamper-evidencing element, as well as methods of making and using the same.

### BACKGROUND

The article “The Fentanyl Patch Boil-Up—A Novel Method of Opioid Abuse (Schauer, C. et al. Basic & Clinical Pharmacology and Toxicology 18 Apr. 2015) states “reports have documented deaths after intravenous (IV) misuse of fentanyl which had been extracted from Duragesic (liquid reservoir type) patches,” and reports “a case of IV fentanyl abuse” related to extraction of fentanyl from a “matrix type” patch.

WO2013/142339 reports a system for monitoring compliance with a medication regimen. Also provided is a system for monitoring compliance with a medication regimen, including a transdermal therapeutic system that has an adhered substrate and a medication. The transdermal therapeutic system is adhered to or affixed to a label comprising an antenna and control electronics. The adherent substrate is configured to be removably attached to a user's body thereby allowing administration of the medication; the label antenna is configured to transmit data. The data transmitted by the antenna may comprise any information representative of, or associated with, the system or the user. Specified data includes a unique or quasi-unique identifier of the transdermal therapeutic patch, the transdermal therapeutic system and/or the formulation disposed within the patch. The data may also be transmitted using magnetic induction.

The article “Controllably degradable transient electronic antennas based on water-soluble PVA/TiO₂ films” (Xu, F. et al. J. Mat. Sci., 53:4, 2638-47, February 2018) discloses antennas made of a composite of PVA and titania nanoparticles that possess excellent radiation performances and that can be physically degraded when immersed in water.

The article “A Physically Transient Form of Silicon Electronics, with Integrated Sensors, Actuators and Power Supply” (Hwang, S.-W. et al. Science 2012 Sep. 28; 337(6102); 1640-4644) discloses silicon electronics that exist for medically useful timeframes before resorption by the body.

U.S. Pat. No. 8,978,452 describes a sensor comprising an RF circuit wherein two termini of the circuit are connected by a jumper including a frangible link. The impedance or resistance of the RF circuit changes by at least a factor of 5 when the frangible link is contacted by a target fluid.

### SUMMARY

A patch can include one or more active pharmaceutical ingredients, at least one tamper-evidencing element having a first state and a second state, the tamper-evidencing element being configured to change from the first state to the second state upon exposure to at least one type of tampering, and at least one antenna having one or more electronically readable features. The electronically readable feature is able to provide a first readout associated with the first state of the tamper-evidencing element and at least a second readout associated with the second state of the tamper-evidencing element, wherein the second readout is differentiable from the first readout. A method of determining whether a patch was exposed to one or more tampering conditions, comprising reading one or more of the at least one electrically readable features and determining whether the readout provided by the patch is the first readout or the second readout.

### BRIEF DESCRIPTION OF THE DRAWINGS

FIG. 1 is a schematic of an antenna with a tamper-evidencing element;

FIG. 2 is a schematic of another antenna with a tamper-evidencing element;

FIG. 3 is a schematic of an antenna that is electronically connected to a circuit with a tamper-evidencing element;

FIG. 4 is a schematic of an antenna that is electronically connected to another circuit with a tamper-evidencing element;

FIG. 5 is a schematic of an antenna that is electronically connected to a circuit with a tamper-evidencing element and a transponder chip;

FIG. 6 is a schematic of an antenna that is electronically connected to another circuit with a tamper-evidencing element and a transponder chip;

FIG. 7A is a schematic of an antenna that is electronically connected to a circuit with a tamper-evidencing element and a sensing chip;

FIG. 7B is a schematic of an antenna that is electronically connected to another circuit with a tamper-evidencing element and a sensing chip; and

FIG. 7C is a schematic of an antenna that is electronically connected to yet another circuit with a tamper-evidencing element and a sensing chip.

### DETAILED DESCRIPTION

Throughout this disclosure, singular forms such as “a,” “an,” and “the” are often used for convenience; however, the singular forms are meant to include the plural unless the singular alone is explicitly specified or is clearly indicated by the context. When the singular alone is called for, the term “one and only one” is typically used.

Some terms in this disclosure are defined below. Other terms will be familiar to the person of skill in the art and should be afforded the meaning that a person of ordinary skill in the art would have ascribed to them.

The terms “common,” “typical,” and “usual,” as well as “commonly,” “typically,” and “usually” are used herein to refer to features that are often employed in this disclosure and, unless specifically used with reference to the prior art, are not intended to mean that the features are present in the prior art, much less that those features are common, usual, or typical in the prior art.

Tampering with drug delivery devices, such as transdermal patches, can involve removing or attempting to remove the pharmaceutically active ingredient or ingredients from the patch. This can be done in order to subsequently administer them by a different mode of administration, such as intravenous administration. Tampering may not only injure the tamperer, who may seek to administer the drug by an unapproved route of delivery, but may give rise to liability for the manufacturer, distributor, or seller of the patch, or for the physician who prescribed the patch.

One way to counter tampering is to determine whether a patch has been tampered with. When the user is required to return a used patch, for example to a pharmacy or dispensary, before obtaining a new patch, then different dosing options can be explored if the return location is able to readily and quickly determine that a patch was exposed to tampering. Ideally it is difficult or impossible to circumvent the feature that allows for determination of whether the user has tampered with the patch, though this is not necessary for tampering counter-measures to be useful or effective.

Thus, a problem to be solved is to provide a patch having features that evidence exposure to one or more tampering conditions. Particular tampering conditions that are applicable to this disclosure are exposure to solvent, which may be any solvent for the pharmaceutically active ingredient in the patch but is most commonly water or a water alcohol mixture. A related problem to be solved is to provide a patch having features that evidence exposure to an attempt, either successful or unsuccessful, to extract the active pharmaceutical ingredient with a solvent, such as water or water alcohol mixtures. Another problem to be solved is to provide features that evidence tampering, particularly tampering by way of an extraction attempt, and have the additional property of not being easily circumvented by a patch user. A related problem to be solved is providing a patch having at least one tamper-evidencing features that are easily readable, particularly electronically readable. A variation on the foregoing is the problem of providing a patch where the at least one tamper-evidencing features can be read without the need for a specialized reader, and in particular cases where the state of the at least one tamper-evidencing element can be read by a common communication device such as a smart phone. A problem to be solved is providing a patch having near-field communication ability that enables a user to determine whether the patch has been exposed to one or more tampering conditions.

Briefly, a solution to one or more of the foregoing problems involves a patch comprising one or more active pharmaceutical ingredients and at least one tamper-evidencing element. The at least one tamper-evidencing element has a first state and a second state and is configured to change from the first state to the second state upon exposure to at least one type of tampering. Most often, the type of tampering is exposure to a solvent, which can be any solvent for the pharmaceutically active ingredient in the patch but is most commonly water or a water alcohol mixture. The patch also includes at least one antenna having one or more electronically readable features, wherein the electronically readable feature provides a first readout associated with the first state of the at least one tamper-evidencing element and at least a second readout associated with the second state of the at least one tamper-evidencing element. The second readout is differentiable from the first readout. The antenna is advantageously a near-field antenna that has the ability to communicate by electronic communication, which is more advantageously a form of RF communication and most advantageously near-field electronic communication.

The patch itself can be any transdermal patch suitable for delivering a pharmaceutically active ingredient. Examples of such patches that are known in the art include those disclosed in, for example, U.S. Pat. No. 9,375,510, WO2016100708, WO200202617, U.S. Pat. Nos. 6,024,976, and 6,149,935. Particular patches include so-called matrix type patches. Other suitable patches may contain microneedle arrays, examples of which are disclosed in U.S. Pat. Nos. 9,144,671, 8,057,842, 7,846,488, 9,789,299, U.S. Ser. No. 10/702,58713, and US20080108958.

The antenna and at least one tamper-evidencing element can be disposed on any suitable portion of the patch, and their location can be determined depending on the configuration of the patch being employed. Suitable portions of the patch include those portions that would expose the at least one tamper-evidencing element to one or more tampering conditions. One common mode of tampering is exposing a patch to a solvent, most often water or a mixture of water and alcohol, to extract the pharmaceutically active ingredient from the patch. Thus, suitable locations for the at least one tamper-evidencing element include those where it would be exposed to a solvent during an attempted extraction of the pharmaceutically active ingredient from the patch. It can be advantageous to place the tamper evidencing element in locations where it does not change from a first state to a second state upon exposure to water or solvents under conditions other than tampering conditions, such as bathing, perspiring, and the like. Such a location can be, for example, between a pharmaceutically active ingredient containing layer of the patch and a backing layer of the patch.

In one exemplary configuration, the patch comprises at least one active pharmaceutical ingredient layer that contains at least one active pharmaceutical ingredient disposed, either directly or through intervening layers, on a substrate. The substrate has at least one backing layer opposite the active pharmaceutical ingredient layer. In this configuration, the tamper-evidencing element can be disposed between the at least one backing layer and the at least one active pharmaceutical ingredient layer.

In another exemplary configuration, the patch comprises an active pharmaceutical ingredient layer that contains both the at least one active pharmaceutical ingredient and the at least one tamper-evidencing element.

In any configuration, including both the two above-mentioned configurations and others, the antenna can be in the same location as the at least one tamper-evidencing element or in a different location. The antenna can also contain the tamper-evidencing device. Regardless, the antenna is configured so that the one or more electronically readable features of the antenna provide a first readout associated with the first state of the at least one tamper-evidencing element and at least a second readout associated with the second state of the at least one tamper-evidencing element.

The at least one tamper-evidencing element can include any element that changes from a first state to a second state upon exposure to at least one type of tampering, most commonly upon exposure to an attempted solvent extraction, which is typically attempted with water or a water alcohol mixture, but in principle can be attempted with other solvents in which the pharmaceutically active ingredient is soluble, which, depending on the identity of the pharmaceutically active ingredient may including acetone, butyl acetate, ethyl acetate, methyl ethyl ketone, or the like, in addition to water or water alcohol mixtures. The first state of the at least one tamper-evidencing element is typically the state of the tamper-evidencing element prior to exposure of the patch to one or more tampering conditions. In many cases, this state is set during the manufacture of the tamper-evidencing element or the patch, but this is not necessary in all cases because a physician, pharmacist, or user could adjust the tamper-evidencing element prior to use. The second state of the tamper-evidencing element is typically a state that occurs after some change to the tamper-evidencing element during exposure to one or more tampering conditions as part of exposure to one or more tampering conditions. The tamper-evidencing element can be a dissolvable or degradable element, in which case it typically dissolves or degrades upon exposure to one or more tampering conditions, such as an attempted extraction with solvent, for example water or water alcohol mixture. It is also possible for the tamper-evidencing agent to dissolve or, more commonly, degrade, in response to physical forces, such as scraping, that are associated with tampering but not with normal use of the patch. The at least one tamper-evidencing element can be a swellable element, such as a swellable gel, typically a swellable hydrogel or superabsorbent polymer, in which case it swells upon exposure to one or more tampering conditions, such as an attempted extraction with solvent, for example water or water alcohol mixture. It is also possible to use at least two tamper-evidencing elements, which can be any combination of the tamper-evidencing elements discussed herein or other tamper-evidencing elements.

In some cases, the tamper-evidencing element can be a component of the antenna, such that exposure to solvent imparts a physical change to the tamper-evidencing element that in turn changes a property of the antenna that can be readout, such as resistance, capacitance, resonance properties such as quality factor (which is sometimes known in the art as Q factor) or resonance frequency, or impedance. The at least one electronically readable feature can be any feature or property of the antenna that can be read by electromagnetic means and can be altered by a change in one or more tamper-evidencing elements. The feature can be, for example, resistance, such as the inherent resistance of the antenna or the resistance of a circuit or circuit element that is connected to the antenna, capacitance, such as the inherent capacitance of the antenna or the capacitance of an element of a circuit connected to the antenna, quality factor, quality factor of the antenna, impedance, such as the inherent impedance of the antenna or the impedance associated with a circuit or circuit element that is electronically connected with the antenna.

The first readout is typically the type or value of the at least one electronically readable feature when the tamper-evidencing element is in the first state. The first readout is, in most cases, the value or type of the at least one electronically readable feature. For example, the first readout can be the value of the resistance, such as the inherent resistance of the antenna or the resistance of a circuit or circuit element that is connected to the antenna, capacitance, such as the inherent capacitance of the antenna or the capacitance of an element of a circuit connected to the antenna, quality factor, such as the quality factor of the antenna, impedance, such as the inherent impedance of the antenna or the impedance associated with a circuit or circuit element that is electronically connected with the antenna. In many cases, the first readout is known to a manufacturer, pharmacy or pharmacist, health care professional, or the like, prior to use of the patch. In some cases, the first readout may be known to the user prior to use of the patch, though this is less common.

The second readout is typically the type or value of the at least one electronically readable feature when the tamper-evidencing element is in the second state. The second readout is, in most cases, the value or type of the at least one electronically readable feature. For example, the second readout can be the value of the resistance, such as the inherent resistance of the antenna or the resistance of a circuit or circuit element that is connected to the antenna, capacitance, such as the inherent capacitance of the antenna or the capacitance of an element of a circuit connected to the antenna, quality factor, such as the quality factor of the antenna, impedance, such as the inherent impedance of the antenna or the impedance associated with a circuit or circuit element that is electronically connected with the antenna. The second readout can be a predetermined value, but that is not necessary so long as it can be determined whether a particular readout is the first readout or the second readout.

For instance, a typical antenna can be in the shape of a coiled wire. Such an antenna can have an intrinsic resistance that depends on the shape of the antenna and the identity of the materials that make up the antenna. An intrinsic impedance and quality factor of the antenna can also be associated with the antenna. An intrinsic capacitance of the antenna can depend on the shape of the coils and the distance between the coils. Each of these features, resistance, impedance, quality factor, and capacitance, can be electronically determined and read out, for example using common electronic monitoring equipment such as a voltmeter, ohmeter, multimeter, impedance analyzer, vector network analyzer, or the like.

One or more of the inherent resistance, impedance, quality factor, and capacitance can be made variable, which can be represented in a circuit diagram as a variable resistor, capacitor, variable impedance element, or the like. To do this, an antenna can be manufactured to be partially or completely composed of a degradable or dissolvable electronic material, which can be, among other things, polyvinylalcohol, such as the polyvinylalcohol titania composites discussed by Xu, F. et al. in J. Mat. Sci., frangible papers such as tissue paper or newsprint; vegetable natural polymers such as alginic acid and alginic acid derivated polymers, arabinogalactan, cellulose derivatives including but not limited to hydroxyethylcellulose, hydroxypropylcellulose, hydroxylpropyl methylcellulose, methylcellulose, carboxymethylcellulose, starch, and starch derivitives; microorganism-derived natural polymers such as polysaccharides, polymers derived from animals including gelatin, collagen, mucopolysaccharides and the like; plyoxyalkylenes; polymers and copolymers derived from ethnically unsaturated monomers including, but not limited to vinylic monomers, acrylates and methacrylates, acrylamides and methacrylamides, and the like; polyethyleneimines; polylactic acid; polyglycolic acid; and mixtures including one or more of the forgoing. Additional suitable substrate materials include polyethylene oxide or polyethylene glycol, pectin, pullulan, and carbopol-based polymer films. Still other suitable substrate materials may be selected from the materials disclosed in PCT publication WO 02/092049 (Godbey et al.), “System for Delivering Cosmetics and Pharmaceuticals”, incorporated herein by reference. Another suitable substrate material is Water-Soluable Wave Solder Tape #5414 available from 3M Company, which is a tape having a PVA film backing, a synthetic water soluble adhesive, and a Kraft paper liner. Additional suitable materials include magnesium, magnesium oxide, silicon oxide, silicon, and silk. Polyvinyl alcohol and polyvinyl alcohol titania composites are most often employed.

The dissolvable or degradable electronic material can act as a tamper-evidencing element. Degradation or dissolution of the polyvinylalcohol, for example in an attempt to tamper with the patch by extracting the active pharmaceutical ingredient with a solvent, such as water or a water alcohol mixture, would therefore change one or more of the resistance, capacitance, impedance, and quality factor of the antenna.

The resistance can be changed, for example, when a portion of the tamper-evidencing element comprise a conductive material disposed on polyvinyl alcohol. In this case, degradation or dissolution of the polyvinyl alcohol breaks the conductive link thereby increasing the overall resistance. The capacitance can be changed, for example, when a portion of the tamper-evidencing element is a pair of parallel metallic plates with a layer of polyvinyl alcohol between the plates. In this case, degradation or dissolution of the polyvinyl alcohol caused by exposure to a solvent under tampering conditions can move the plates closer together, thereby changing the capacitance. Alternatively, a swelling material, such as a superabsorbent polymer, can be between the plates. In that case, swelling of the polymer caused by exposure to a solvent under tampering conditions can move the plates farther apart, thereby changing the capacitance. Impedance is a function of the resistance, capacitance, and inductance of a circuit, so changing resistance or capacitance as described herein will also change the impedance.

The value of one or more of the resistance, impedance, and quality factor prior to exposure to one or more tampering conditions, such as the value at the time of manufacture, the time of sale or transfer to the user, or the like, can be the first state of the tamper-evidencing element, and can be provided as the first readout. The value of one or more of the resistance, impedance, and quality factor after exposure to one or more tampering conditions, when all or part of the dissolvable or degradable material has been dissolved or degraded, for example by an extraction or attempted extraction by solvent such as water or a water alcohol mixture, can be the second state of the tamper-evidencing element. The value of one or more of the resistance, impedance, and quality factor can be provided as a second readout.

Another configuration involves placing a dissolvable or degradable material, such as those discussed herein, or a swellable material, such as a superabsorbent polymer, which can be any superabsorbent polymer known to the artisan for example sodium polyacrylate, between the coils of the antenna. The antenna coils with the dissolvable or degradable material or the swellable material can then act as a tamper-evidencing element. The capacitance of the antenna in this configuration, which can be the first state of the tamper-evidencing element, can be provided as a first readout. After exposure to one or more tampering condition, such as an extraction attempt with a solvent, for example water or a water alcohol mixture, the geometry of the antenna coils can change. In the case that the coils were being held apart by a dissolvable or degradable substance, they can move closer together after dissolution or degradation of the substance; in the case that a swellable material is between the coils, swelling of the material can push the coils farther apart. In either case, the new configuration can be the second state of the tamper-evidencing element, and the capacitance of the antenna will be different in the second state than in the first state (i.e., before dissolution, degradation, or swelling, as the case may be, associated with the one or more tampering condition). The capacitance in the second state can be provided as a second readout.

The antenna can also be electronically connected to a circuit having one or more of a variable resistor, variable capacitor, variable impedance, and variable quality factor, which can act at the tamper-evidencing element. In particular cases, the resistance or impedance of the circuit changes by at least a factor of 5 when the tamper-evidencing element is contacted by a solvent, such as water or a water alcohol mixture.

A variable resistor can be constructed by making all or part of the resistor from a degradable, dissolvable, or swellable material, such as those discussed above. When a degradable or dissolvable material is used, then upon degradation or dissolution the resistance of the variable resistor will change with the resistor geometry. It is also possible that the entire variable resistor degrades, leaving an open circuit. A change in geometry and related change in resistance can also be achieved using a swellable material, such as a superabsorbent polymer. It is also possible to dispose a resistor adjacent to a superabsorbent polymer so that the resistor will break upon swelling of the superabsorbent polymer. In any of those cases a first readout can be the resistance prior to exposure to tampering conditions.

When a circuit is electronically connected to the antenna, a variable capacitor can be a component of the electronic circuit to act as a tamper-evidencing element. A variable capacitor can have a swellable material, such as a superabsorbent polymer, disposed between two plates of the capacitor. This can be the first state, in which the capacitance can be provided as the first readout. Upon exposure to one or more tampering conditions, such as exposure to a solvent for example water or water alcohol mixtures, the swellable material can swell thereby increasing the distance between the two plates of the capacitor and changing the capacitance; it is also possible that the swellable material swells and physically breaks the capacitor. In either case, the state where the swellable material has swelled can be the second state, and the capacitance after swelling can be provided as the second readout. Alternatively, a variable capacitor can be made at least in part from a dissolvable or degradable material, in which case the first state can be the state when the dissolvable or degradable material is intact and the first readout can be the capacitance associated with that state. The second state can be the state when the dissolvable or degradable material has dissolved or degraded, and the capacitance associated with the second state can be provided as a second readout.

In any of the above cases, a chip, such as a transponder chip, can be electronically connected to the antenna or, if present, the circuit. The chip, which is usually a transponder chip, can impart additional functionality, such as providing a unique or quasi-unique serial number, providing manufacturing or expiration information, providing readable or readable/writable memory, for example as may be accomplished by an RFID tag, a Near Field Communication (sometimes known as NFC) tag, a tag imparting near field communication functionality, or the like. In many cases, the chip is a sensing chip that can detect one or both of the first state and second state of the tamper-evidencing element. In some cases, the sensing chip, which can optionally be part of an integrated circuit, can also include an analog to digital converter.

Exemplary chips can detect one or more of capacitance, resistance, and impedance. When the chip is a transponder chip, it can also facilitate reading out of one or more of the first and second readout. Suitable chips can include those known as NFC sensing integrated circuits, such as the chip sold under the trade designation NHS3152 as well as those known as tag tamper detectors, such as the chip sold under the trade designation NTAG213TT, both of which are available from NXP Semiconductors N.V. (Eindhoven, Netherlands).

The tamper-evidencing features as described above are difficult to circumvent, particularly in matrix-type patches, wherein the drug cannot readily be removed by scraping but can be extracted by exposure to solvent, such as water or water alcohol mixtures. By locating the tamper-evidencing features in a location as described herein where a tampering attempt, particularly an attempt to extract active pharmaceutical ingredient with solvent, would necessarily expose the tamper-evidencing element to the solvent, it would be very difficult to avoid changing the tamper-evidencing element from a first state to a second state. If the tamper-evidencing element were removed from the patch, the removal could be detected. In this case, the inability to detect the capacitance, impedance, resistance, or the like of the tamper-evidencing element because of its removal could be considered the second readout. The second readout would be different from the first readout if the tamper-evidencing element were removed. Thus, the removal of the chip, which would presumably be associated with a tampering condition, can be detected.

In use, a method can involve determining whether a patch as described herein has been exposed to one or more tampering conditions, such as an attempted extraction by a solvent, particularly water or a water alcohol mixture. The method can comprise reading one or more of the at least one electrically readable features of a patch and determining whether the readout provided by the patch is the first readout or the second readout. In particular cases, especially those where the patch includes a near-field antenna, a transponder chip that enables near-field communication, or both, the step of reading one or more of the at least one electrically readable features of the patch can involve receiving a near-field electronic signal from the patch.

In an exemplary use, one or more patches with tamper-evidencing elements as described herein can be issued to a patch user, for example, by a pharmacy. After using the patches, the user can be required to return the used patches to the pharmacy before new patches are issued. The patches have a first readout that is known to the pharmacy. For example, when the patch is equipped with near-field communication capabilities, the pharmacy can use a smart-phone or other device with near-field communication capabilities to detect the first readout at the time that the patch is dispensed; alternatively, the first readout can be communicated to the pharmacy from the manufacturer or distributor of the patch.

Upon returning the patches, the pharmacy can use a smart phone or other near-field communication device to read one or more electronically readable features of the tamper-evidencing device. Comparing of the electronically readable feature that is read by the pharmacy to the known first readout allows the pharmacy to determine whether the first readout or the second readout was read. A first readout indicates that the patch has not been exposed to tampering conditions and the patch user can therefore be issued one or more additional patches. A second readout indicates that the patch has been exposed to tampering conditions, which can signal the pharmacy to take appropriate action, such as exploring other dosing options.

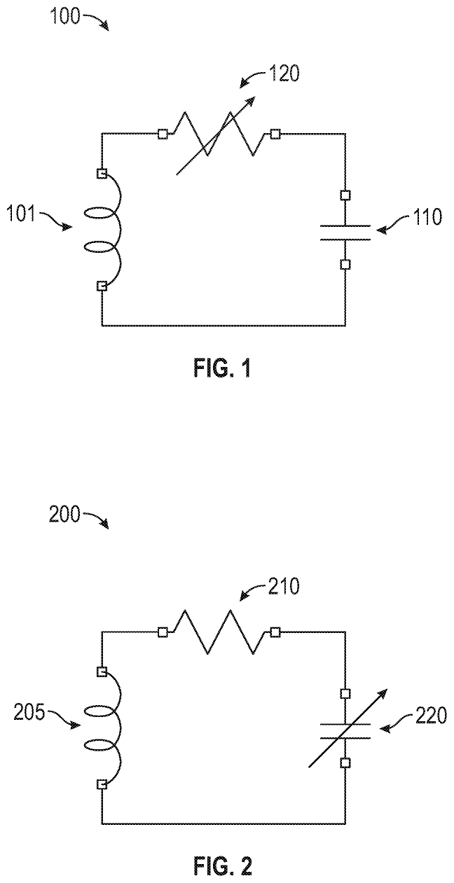

Turning to the drawings, FIG. 1 shows antenna 100, which includes metal coil 101, an intrinsic capacitance depending on, among other things the distance between the coils, which act as a capacitor 110 as well as variable resistor 120, which is made in part of a material that changes in resistance upon exposure to solvent. For example, the antenna can be made in part out of a dissolvable or degradable material that dissolves or degrades upon exposure to tampering conditions such as extraction with a solvent, typically water or a water alcohol mixture. The change in geometry that results from exposure degradation or dissolution of the degradable or dissolvable material also provides a change in resistance. As another example, a swellable material such as a superabsorbent polymer can be incorporated into the antenna to change the geometry upon exposure to solvent. In these or other cases where the resistance changes, the initial resistance is the first state and the changed resistance is the second state. When the first readout of the antenna associated with the first state of the tamper-evident element, such as the antenna's resistance, is known, then it can be readily determined whether the antenna provides the first readout corresponding to the first state of the tamper-evidencing element or a second readout corresponding to the second state of the tamper-evidencing element.

FIG. 2 shows antenna 200, which includes metal coil 205 and which has an intrinsic resistance as shown by intrinsic resistor 210 as well as a variable capacitor 220. Variable capacitor 220 can be configured as the tamper-evidencing element in that the capacitance can change upon exposure to solvent, such as water or a water alcohol mixture. For example, when variable capacitor 220 is the coils of the antenna 200, the coils can be separated by a dissolvable solid, which can be any dissolvable solid but is commonly a polymer such as a water-soluble acrylate or acrylamide that is dissolvable in water or water alcohol mixtures, that pushes the coils apart thereby maintaining the distance between the coils. Upon exposure to solvent such as water or water alcohol mixtures the coils of the antenna can move closer together in the same manner as a stretched spring after the tension is released, thereby changing the capacitance. Alternatively, the coils can be separated by a superabsorbent polymer, which can be any superabsorbent polymer known to the artisan for example sodium polyacrylate, that swells upon exposure to solvent, such as water or a water alcohol mixture, thereby increasing the distance between the coils and changing the capacitance. In either case, the initial capacitance can be the first state of the tamper-evidencing element and the capacitance after exposure to solvent can be the second state of the tamper-evidencing element. When the first readout of the antenna associated with the first state of the tamper-evident element, such as the antenna's capacitance, is known, then it can be readily determined whether the antenna provides the first readout corresponding to the first state of the tamper-evidencing element or a second readout corresponding to the second state of the tamper-evidencing element.

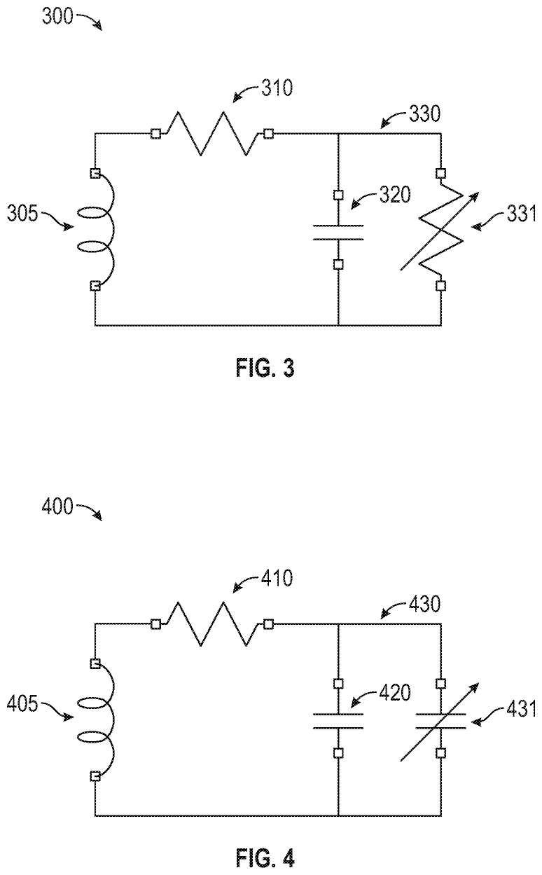

FIG. 3 depicts antenna 300, which has a metal coil 305 and an intrinsic resistance represented by resistor 310 and an intrinsic capacitance represented by capacitor 320. The antenna is electronically connected to circuit 330 containing variable resistor 331 which functions as a tamper-evidencing element. The variable resistor 331 can be in the form of a metal trace attached to the antenna's endpoints and can act as the tamper-evidencing element, for example, in an analogous manner to the variable resistance described above with respect to the resistor of FIG. 1. Alternatively, the variable resistor 331 can be designed to completely dissolve or degrade upon exposure to solvent, such as water or a water alcohol mixture. In another variation, the variable resistor 331 can be adjacent to or on a swellable material, such as a superabsorbent polymer, so that the variable resistor 331 breaks when the swellable material swells. In any of these variations, the resistance of the circuit changes upon exposure to one or more tampering conditions, specifically an extraction attempt using solvent, typically water or a water alcohol mixture.

FIG. 4 features antenna 400 which has a metal coil 405 and an intrinsic resistance represented by resistor 410 and an intrinsic capacitance represented by capacitor 420. The antenna is connected to circuit 430 containing variable capacitor 431, which functions as a tamper-evidencing element because its geometry or dielectric constant changes from the first state, which is the initial state before exposure to one or more tampering conditions, to a second state in response to one or more tampering condition. For example, variable capacitor 431 can be in the form of a metal trace attached to the antenna's endpoints and can act as the tamper-evidencing element by placing a dissolvable or degradable element between the plates (not shown) of capacitor 431 such that the distance between the plates changes upon exposure to one or more tampering conditions, such as exposure to solvent such as water or a water alcohol mixture. Alternatively, variable capacitor 431 can have a swellable material, such as a superabsorbent polymer (not shown), disposed between the plates such that the distance between the plates increases when the swellable material is exposed to solvent such as water or a water alcohol mixture. In another variation, capacitor 431 can completely or partially dissolve or degrade upon exposure to one or more tampering conditions, such as exposure to solvent such as water or a water alcohol mixture. In yet another variation, capacitor 431 can be designed to physically break when the swelling material swells after exposure to one or more tampering conditions, such as exposure to solvent such as water or a water alcohol mixture.

In FIGS. 3 and 4, the first state of the tamper-evidencing element is the state when the element is not dissolved, degraded, or swelled, and the second state of the tamper-evidencing element is the state when the element is dissolved, degraded, or swelled. The first readout of the antenna can relate to the resistance, capacitance, or impedance of the circuit in the first state, when no dissolution, degradation, or swelling of the tamper-evidencing element has occurred. The second readout of the antenna can relate to the resistance, capacitance, or impedance of the circuit in the second state, after swelling, dissolution, or degradation has occurred.

In a variation of the circuits shown in either FIG. 3 or FIG. 4, one or more circuit feature that varies the impedance of the circuit upon exposure to one or more tampering conditions, such as an attempted extraction by solvent for example water or a water alcohol mixture, can be employed in addition to or instead of the variable resistor or variable capacitor. The one or more circuit features can include circuit features that have a dissolvable, degradable, or swellable component that changes the impedance of the circuit upon dissolution, degradation, or swelling. In such cases, the circuit feature can act as a tamper-evidencing element. The circuit feature prior to dissolution, degradation, or swelling can be the first state of the tamper-evidencing element and the circuit feature after dissolution, degradation, or swelling can be the second state of the tamper-evidencing element. Thus, the impedance prior to dissolution, degradation, or swelling can be the first readout and the impedance prior to dissolution, degradation, or swelling can be the second readout.

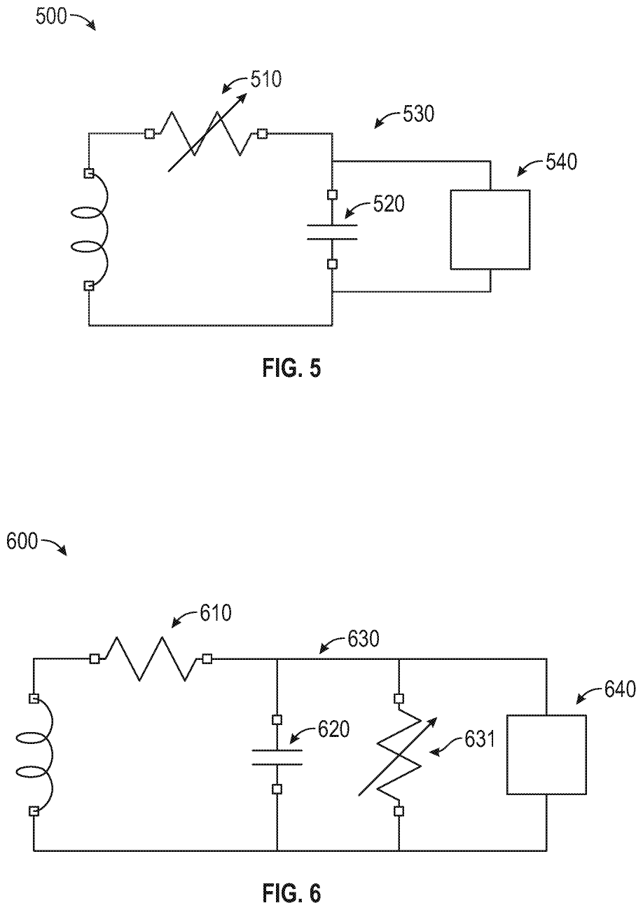

FIG. 5 shows antenna 500 featuring variable resistor 510 that functions as the tamper-evidencing element and is similar to variable resistor 120, and having an intrinsic capacitance represented by capacitor 520. Antenna 500 is electronically connected to circuit 530 which features transponder chip 540. Antenna 500 functions in much the same manner as antenna 100, discussed herein. The addition of transponder chip 540 permits additional features, such as providing a unique or quasi-unique serial number, providing manufacturing or expiration information, providing readable or readable/writable memory, for example as may be accomplished by an RFID tag, imparting near field communication functionality, or the like. The first and second state of the tamper-evidencing element, i.e. variable resistor 510, as well as the associated first and second readouts, can be similar to those discussed above with respect to FIGS. 1 and 3.

Variations of FIG. 5 with a variable capacitor or a variable impedance, either alone or in combination and either in addition to or instead of the variable resistor, are also contemplated.

FIG. 6 shows antenna 600 featuring intrinsic resistor 610 and having an intrinsic capacitance represented by capacitor 620 and is electronically connected to circuit 630. Circuit 630 features variable resistor 631 and includes transponder chip 640. Antenna 600 thus functions in a similar manner to antenna 300, discussed herein, but with the additional functionality imparted by transponder chip 640. That additional functionality is similar to that discussed herein with regard to transponder chip 540 herein.

Variations of FIG. 6 with a variable capacitor or a variable impedance, either alone or in combination and either in addition to or instead of the variable resistor, are also contemplated. Variations of FIG. 6 wherein one or more of resistor 610, capacitor 620, are variable are also contemplated, as are variations with variable impedance; in any of the above variations variable resistor 631 may be replaced with a standard resistor.

The first and second state of the tamper-evidencing element, i.e. variable resistor 631, as well as the associated first and second readouts, can be similar to those discussed above with respect to FIG. 5.

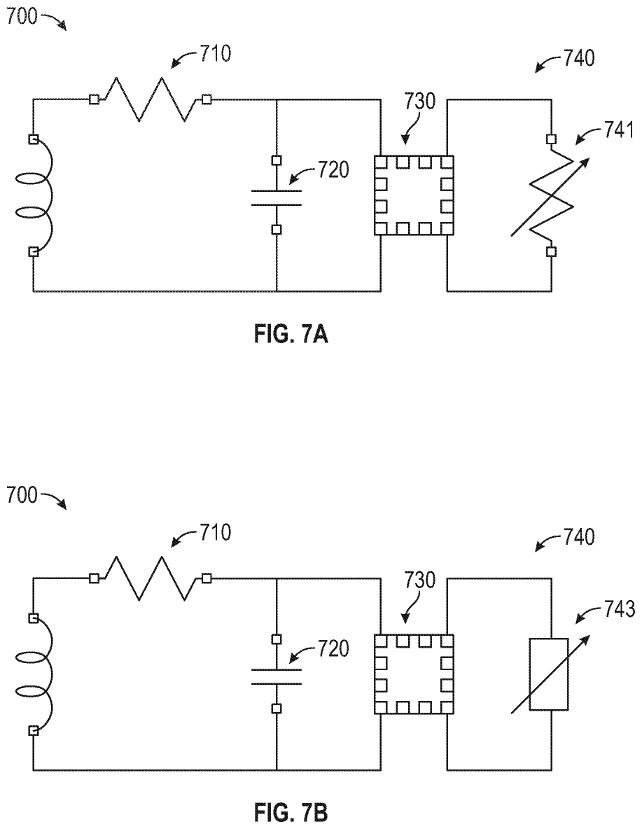

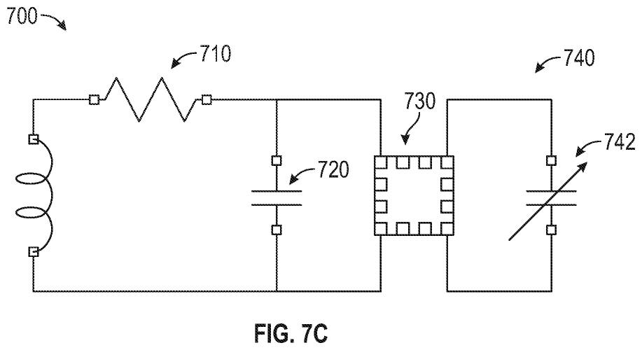

FIGS. 7A, 7B, and 7C feature antenna 700 having intrinsic resistor 710 and intrinsic capacitor 720, both of which are intrinsic to the antenna. Antenna 700 is electronically connected to circuit 740 which contains chip 730. Chip 730, which can be any type of chip suitable for circuit 740, is in this case a sensing chip that, in addition to the capabilities associated with transponder chips such as those described above with respect to transponder chip 540, can have the capability to measure impedance, capacitance, or both, as well as resistance.

In FIG. 7A, chip 730 can be electronically connected to a variable resistor 741, which can function in a similar manner to, for example, other variable resistors described herein. Chip 730 can detect or measure resistance. When chip 730 is a transponder chip, it can facilitate reading out the first and second readouts, which can relate, respectively, to the resistance of variable resistor 741 before and after exposure to one or more tampering conditions, such as extraction or attempted extraction by a solvent.

FIG. 7B is similar to FIG. 7A but has a device 743 which can be a tamper-evidencing element that imparts impedance to the circuit. The impedance can change upon exposure to one or more tampering conditions. Chip 730 can detect or measure impedance and, when it is a transponder chip, can facilitate reading out the first and second readouts, which can relate, respectively, to the impedance or quality factor of circuit 740 before and after exposure to one or more tampering conditions, such as extraction or attempted extraction by a solvent.

FIG. 7C is similar to FIGS. 7A and 7B but features variable capacitor 742 which can be a tamper-evidencing element and can function in a similar manner to other variable capacitors described herein. Chip 730 can detect or measure capacitance and, when it is a transponder chip, can facilitate reading out the first and second readouts, which can relate, respectively, to the capacitance or stored electrical energy in integrated circuit 740 before and after exposure to one or more tampering conditions, such as extraction or attempted extraction by a solvent.

1. A patch comprising:
one or more active pharmaceutical ingredients;
at least one tamper-evidencing element having a first state and a second state, the tamper-evidencing element being configured to change from the first state to the second state upon exposure to at least one type of tampering; and
at least one antenna having one or more electronically readable features, wherein the electronically readable feature is able to provide a first readout associated with the first state of the tamper-evidencing element and at least a second readout associated with the second state of the tamper-evidencing element, wherein the second readout is differentiable from the first readout.

2. The patch of claim 1, wherein the patch comprises at least one active pharmaceutical ingredient layer on a substrate having at least one backing layer opposite the active pharmaceutical ingredient layer; wherein
the tamper-evidencing element is disposed between the at least one backing layer and the at least one active pharmaceutical ingredient layer.

3. The patch of claim 1, wherein the patch comprises at least one active pharmaceutical ingredient layer comprising at least one active pharmaceutical ingredient and the tamper-evidencing element.

4. The patch of claim 1, further comprising a circuit that is electronically connected to the antenna, the circuit comprising a transponder chip for storing and transmitting at least one piece of information.

5. The patch of claim 4, wherein at least one piece of information is a first electronically readable feature when the tamper-evidencing element is in the first state and a second piece of information when the tamper-evidencing element is in the second state.

6. The patch of claim 4, wherein the at least one piece of information is either (1) associated with the identity of the patch; (2) associated with the identity of a patient to whom the patch is prescribed; or (3) associated with both the identity of the patch and the identity of a patient to whom the patch is prescribed.

7. The patch of claim 1, wherein the antenna is a near-field antenna.

8. The patch of claim 1, wherein the patch further comprises at least one microneedle protruding from a first major surface of the patch.

9. The patch of claim 1, wherein the patch further comprises at least one skin-contact adhesive disposed thereon.

10. The patch of claim 1, wherein the tamper-evidencing element is a dissolvable or degradable element.

11. The patch of claim 1, wherein the tamper-evidencing element is a swellable element.

12. The patch of claim 4, wherein the resistance or impedance of the circuit changes by at least a factor of 5 when the tamper-evidencing element is contacted by a solvent, wherein the solvent is optionally water or a water alcohol mixture.

13. The patch of claim 4, wherein the circuit includes at least one analog to digital converter.

14. The patch of claim 1, wherein the tamper-evidencing element is a component of the antenna.

15. The patch of claim 1, comprising at least two tamper-evidencing elements.

16. The patch of claim 1, further comprising a sensing integrated circuit for sensing patient information or patch information.

17. The patch of claim 1, wherein the at least one pharmaceutically active ingredient is an opioid pharmaceutical.

18. The patch of claim 1, wherein the opioid pharmaceutical is fentanyl.

19. A method of determining whether a patch was exposed to one or more tampering conditions, comprising:
reading one or more of the at least one electrically readable features of the patch of claim 1; and
determining whether the readout provided by the patch is the first readout or the second readout.

20. The method of claim 19, wherein the step of reading one or more of the at least one electrically readable features of the patch comprises receiving a near-field electronic signal from the patch.
